(19) United States
(12) Patent Application Publication
(10) Pub. No.: US 2021/0102953 A1
(43) Pub. Date: Apr. 8, 2021

(54) MASS SPECTROMETRY KIT, MICROORGANISM IDENTIFICATION KIT, SAMPLE PREPARATION METHOD, ANALYSIS METHOD, AND MICROORGANISM IDENTIFICATION METHOD

(71) Applicant: SHIMADZU CORPORATION, Kyoto-shi, Kyoto (JP)

(72) Inventor: Kanae TERAMOTO, Kyoto-shi, Kyoto (JP)

(73) Assignee: SHIMADZU CORPORATION, Kyoto-shi, Kyoto (JP)

(21) Appl. No.: 17/047,997

(22) PCT Filed: Mar. 8, 2019
(86) PCT No.: PCT/JP2019/009392

§ 371 Date: Oct. 15, 2020

(30) Foreign Application Priority Data
Apr. 16, 2018 (JP) 2018-078584

(51) Int. Cl. G01N 33/68; G01N 27/62; H01J 49/04; H01J 49/16; H01J 49/00

(52) U.S. Cl. CPC G01N 33/6851; G01N 27/62; H01J 49/0418; G01N 2333/195; H01J 49/164; H01J 49/0031; H01J 49/0431

(57) ABSTRACT

A mass spectrometry kit for use in mass spectrometry with ionizing a sample by matrix-assisted laser desorption/ionization includes: a matrix reagent in a solid state and/or an additive of matrix in a solid state; and a plurality of containers each containing the matrix reagent and/or the additive therein.

### TECHNICAL FIELD

The present invention relates to a mass spectrometry kit, a microorganism identification kit, a sample preparation method, an analysis method, and a microorganism identification method.

### BACKGROUND ART

Mass spectrometry with ionizing samples by matrix-assisted laser desorption/ionization (hereinafter referred to as MALDI) is an analysis method suitably used for various applications such as identification of microorganisms. In the mass spectrometry using MALDI, a plurality of prepared samples (hereinafter referred to as “mass spectrometry samples”) are placed at a plurality of sample placement sites of a sample plate, and the sample placement sites are irradiated with a laser to ionize the mass spectrometry samples.

In the case of placing dozens or more of mass spectrometry samples on one sample plate and then placing the sample plate in a mass spectrometer for mass spectrometry, a complicated operation in preparation of these mass spectrometry samples is involved.

The use of a suitable additive of matrix in addition to a matrix in preparation of the mass spectrometry samples may result in obtaining more accurate measurement data (see PTL 1). However, the operation of adding the additive of matrix to each of the samples involves a more complicated operation in preparation of the mass spectrometry samples.

### CITATION LIST

### Patent Literature

PTL1: JP 2007-121857 A

### SUMMARY OF INVENTION

### Technical Problem

It is thus desired to reduce the complicated operation in preparation of mass spectrometry samples for matrix-assisted laser ionization/desorption.

### Solution to Problem

According to the 1st aspect of the present invention, a mass spectrometry kit for use in mass spectrometry with ionizing a sample by matrix-assisted laser desorption/ionization, comprises: a matrix reagent in a solid state and/or an additive of matrix in a solid state; and a plurality of containers each containing the matrix reagent and/or the additive therein.

According to the 2nd aspect of the present invention, in the mass spectrometry kit according to the 1st aspect, it is preferred that a capacity of each of the plurality of containers is 100 μL or more to 5 mL or less.

According to the 3rd aspect of the present invention, in the mass spectrometry kit according to the 2nd aspect, a capacity of each of the plurality of containers is 200 μL or more to 3 mL or less.

According to the 4th aspect of the present invention, in the mass spectrometry kit according to any one of the 1st to 3rd aspects, it is preferred that the number of the plurality of containers is 5 or more.

According to the 5th aspect of the present invention, in the mass spectrometry kit according to any one of the 1st to 4th aspects, it is preferred that the plurality of containers each contain a mixture of the matrix reagent and the additive.

According to the 6th aspect of the present invention, in the mass spectrometry kit according to any one of the 1st to 5th aspects, it is preferred that the matrix reagent includes a substance constituting a solid matrix or a liquid matrix.

According to the 7th aspect of the present invention, it is preferred that the the mass spectrometry kit according to any one of the 1st to 6th aspects further comprises: a solvent placed in a solvent container that is different from the plurality of containers each containing the matrix reagent and/or the additive.

According to the 8th aspect of the present invention, in the mass spectrometry kit according to any one of the 1st to 7th aspects, it is preferred that the additive includes a phosphonic acid group-containing compound.

According to the 9th aspect of the present invention, a microorganism identification kit comprises the mass spectrometry kit according to any one of the 1st to 8th aspects.

According to the 10th aspect of the present invention, a sample preparation method for preparing a plurality of samples for use in mass spectrometry with matrix-assisted laser desorption/ionization, comprises: providing a plurality of containers each containing a matrix reagent in a solid state and/or an additive of matrix in a solid state therein; and preparing mass spectrometry samples containing respective samples corresponding to the plurality of containers, using the matrix reagent and/or the additive.

According to the 11th aspect of the present invention, in the mass spectrometry kit according to the 10th aspect, it is preferred that the mass spectrometry samples are prepared by adding the respective samples, the matrix reagent, and the solvent to the plurality of containers each containing the additive therein.

According to the 12th aspect of the present invention, in the mass spectrometry kit according to the 10th aspect, it is preferred that a mixture of the matrix reagent and the additive is placed in each of the plurality of containers, and the mass spectrometry samples are prepared by adding the respective samples and the solvent to the plurality of containers each containing the matrix reagent and the additive therein.

According to the 13th aspect of the present invention, an analysis method comprises: preparing a mass spectrometry sample by the sample preparation method according to any one of the 10th to 12th aspects; irradiating the mass spectrometry sample with a laser to ionize the mass spectrometry sample; subjecting the ionized mass spectrometry sample to mass spectrometry.

According to the 14th aspect of the present invention, a microorganism identification method comprises: preparing a mass spectrometry sample containing a plurality of proteins contained in a microorganism by the sample preparation method according to any one of the 10th to 12th aspects; irradiating the mass spectrometry sample with a laser to ionize the mass spectrometry sample; subjecting the ionized mass spectrometry sample to mass spectrometry to create a mass spectrum; comparing peaks in the mass spectrum with peaks in mass spectra of proteins contained in a plurality of microorganisms stored in a database; and identify what kind of microorganism the microorganism is on the basis of the comparison.

### Advantageous Effects of Invention

The present invention can reduce the complicated operation in preparation of mass spectrometry samples by using a matrix or an additive of matrix, thereby allowing mass spectrometry to be performed efficiently.

### BRIEF DESCRIPTION OF DRAWINGS

FIG. 1 is a conceptual diagram illustrating a mass spectrometry kit according to one Embodiment.

FIG. 2 is a conceptual diagram illustrating a sample preparation method according to the Embodiment.

FIG. 3 is a flowchart illustrating flow of a microorganism identification method according to the Embodiment.

FIG. 4 is a conceptual diagram illustrating a mass spectrometry kit of a Variation.

FIG. 5 is a conceptual diagram illustrating a sample preparation method according to the Variation.

FIG. 6 is a flowchart illustrating a microorganism identification method according to the Variation.

FIG. 7 is a conceptual diagram illustrating a mass spectrometry kit of another Variation.

FIG. 8 is a conceptual diagram illustrating a mass spectrometry kit of yet another Variation.

FIG. 9 shows mass spectra of mass spectrometry samples prepared from normal skin flora without using an additive of matrix in Example 1.

FIG. 10 shows mass spectra of mass spectrometry samples prepared from normal skin flora by using a mass spectrometry kit containing MDPNA as an additive of matrix in Example 1.

FIG. 11 shows a mass spectrum of a mass spectrometry sample prepared from a lactic acid bacterium by using a mass spectrometry kit containing MDPNA as an additive of matrix (upper spectrum) and a mass spectrum of a mass spectrometry sample prepared from a lactic acid bacterium without using an additive of matrix (lower spectrum), in Example 2.

### DESCRIPTION OF EMBODIMENTS

The Embodiments of the present invention will be described below with reference to the drawings.

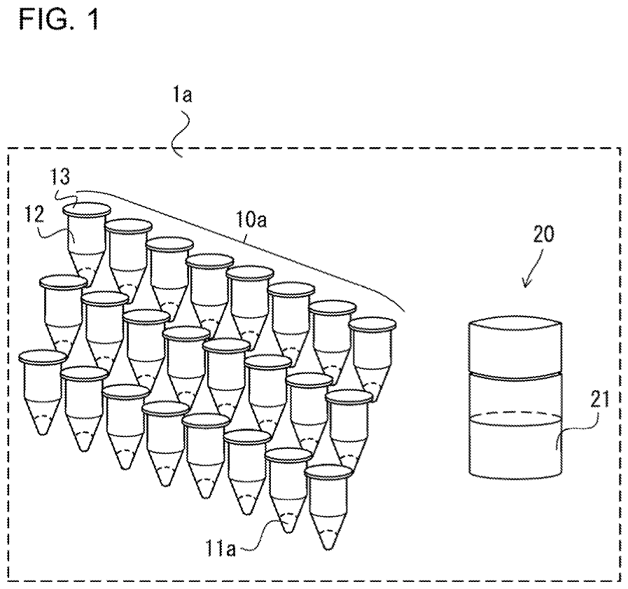

FIG. 1 is a conceptual diagram illustrating a mass spectrometry kit according to this Embodiment. A mass spectrometry kit 1a includes an additive of matrix (hereinafter also merely referred to as “additive”) 11a in the solid state, a plurality of containers (hereinafter referred to as “additive containers”) 10a each containing the additive 11a therein, and a container (herein after referred to as “solvent container”) 20 containing a solvent 21. Each of the additive containers 10a includes an additive container body 12 and an additive container lid 13.

The mass spectrometry kit 1a may further include other substances used in mass spectrometry. The mass spectrometry kit 1a is only required to include additive containers 10a each containing the additive 11a therein, and may not include a solvent 21.

The capacity of each of the additive containers 10a is preferably based on the amount of mass spectrometry sample added dropwise on a site (hereinafter referred to as “sample placement site”) on which a sample is placed in a sample plate at the time of mass spectrometry that performs ionization by the MALDI. This allows each mass spectrometry sample to be suitably prepared by adding a sample and a matrix reagent to each of the additive containers 10a.

It should be noted that a solution (hereinafter referred to as “additive solution”) containing an additive 11a may be prepared in each of the additive containers 10a and then dispensed in other containers, and the mass spectrometry sample may be prepared in each of the containers.

If one kind of mass spectrometry sample is prepared in one additive container 10a, which is then placed at several sample placement sites, several microliters of the mass spectrometry sample are required assuming the 1 μL of the mass spectrometry sample is added dropwise per sample placement site. Even considering that the amount of the mass spectrometry sample placed at a sample placement site may be set to 0.2 μL or 1.5 μL which is different from 1 μL, the capacity of each of the additive containers 10a does not have to be so large. Accordingly, the capacity of each of the additive containers 10a is preferably 5 mL or less, more preferably 2 mL or less, yet more preferably 1.5 mL or less, yet more preferably 1.0 mL or less, yet more preferably 0.5 mL or less, for saving space in storage.

Too small capacity of each of the additive containers 10a causes difficulty in handling. Thus, the capacity of each of the additive containers 10a is preferably 100 μL or more, more preferably 200 μL or more.

Although FIG. 1 shows twenty-four additive containers 10a, the number of additive containers 10a each containing an additive 11a therein included in the mass spectrometry kit 1a is not particularly limited.

A sample plate for MALDI, having, for example, 96 or 384 sample placement sites is commercially available. Once a sample plate is placed in a mass spectrometer, calibration is performed in this state, and then, samples at several to several hundred sample placement sites may be analyzed. Accordingly, the number of additive containers 10a included in the mass spectrometry kit 1a is preferably two or more, more preferably five or more, yet more preferably 10 or more, yet more preferably 20 or more. The greater the number of additive containers 10a included in the mass spectrometry kit 10a, the less operation is required to refill the additive containers 10a each containing an additive 11a by purchasing or the like.

The too great number of additive containers 10a included in the mass spectrometry kit 1a causes problems of losing activity of the additive 11a due to the long storage period, or requires large space for storage, which results in narrow space for other articles. Thus, the number of additive containers 10a can be appropriately 1000 or less, or 500 or less.

The shape or the like of each of the additive containers 10a is not particularly limited, the additive container lid 13 may integrates with the additive container body 12, or the additive container lid 13 and the additive container body 12 may be different parts. The additive containers 10a may be formed to integrate with each other, and for example, the additive container body 12 and/or the additive container lid 13 may integrate per the predetermined number of additive containers 10a such as 8 strip tubes.

The additive 11a placed in each of the additive containers 10a is not particularly limited as long as it is used to prepare a mass spectrometry sample containing a matrix and is expected to exhibit some effect of reducing noises, enhancing detection accuracy for analysis target ions, and the like.

Preferably, the additive 11a includes a phosphonic acid group-containing compound. A compound containing one phosphonic acid group is preferably phosphonic acid, methylphosphonic acid, phenylphosphonic acid, 1-naphthylmethylphosphonic acid or the like.

A compound containing two or more phosphonic acid groups is preferably methylenediphosphonic acid (hereinafter referred to as MDPNA), ethylenediphosphonic acid, ethane-1-hydroxy-1,1-diphosphonic acid, nitrilotriphosphonic acid, ethylenediaminetetraphosphonic acid or the like, more preferably MDPNA.

The mass spectrometry of a mass spectrometry sample prepared using an additive 11a containing a phosphonic acid group-containing compound can reduce noises, enhance detection accuracy of, for example, proteins contained particularly in a cytoplasm component or the like, and enhance detection accuracy of a peptide such as peptide phosphate. These effects are exhibited for both positive ion detection and negative ion detection in mass spectrometry.

The additive 11a in the solid state is stored in each of the additive containers 10a. This allows the additive 11a to remain active for a longer period of time than the case of dissolving the additive 11a in a solvent and stored as a solute.

How to produce additive containers 10a each containing an additive 11a therein is as follows. A solution containing the additive 11a at a predetermined concentration is prepared using a suitable solvent, and the predetermined amount of the solution is dispensed into each of the additive containers 10a with, for example, a pipette or a dispenser. The additive containers 10a into which the solution containing the additive 11a has been dispensed are dried to obtain the additive containers 10a each containing the additive 11a in the solid state therein. How to dry the solution containing the additive 11a is not particularly limited. The additive container lid 13 for the additive containers 10a may be left open, or additionally, the additive containers 10a may be depressurized by, for example, a vacuum dryer.

A solvent 21 stored in a solvent container 20 which is different from the additive containers 10a in the mass spectrometry kit 1a is used appropriately to prepare a solution containing a matrix reagent (hereinafter referred to as “matrix solution”), an additive solution, and other solutions in preparation of a mass spectrometry sample.

The solvent 21 is preferably an organic solvent, or a solvent obtained by mixing an organic solvent and an aqueous solvent, and the kinds of the organic solvent and the aqueous solvent are not particularly limited. As an example, the solvent 21 is an aqueous solution containing trifluoroacetic acid (TFA) that has been prepared to have a predetermined concentration by vol % of, for example, 0% or more to 1% or less and acetonitrile (ACN) having any concentration. The concentration of acetonitrile by vol % can be set appropriately to dozens of percent, particularly 50% or the like. The solution containing a sample, a matrix reagent, and other substances, prepared using the solvent 21 prepared to contain an organic solvent in the above manner can easily dissolve the matrix reagent, and when the sample is, for example, a Gram-negative bacterium, can break cell walls, thereby allowing the mass spectrometry sample obtained to be easily ionized.

The shape, the capacity and the like of the solvent container 20 storing the solvent 21 are not particularly limited, and a container such as a vial in any size can be used as the solvent container 20.

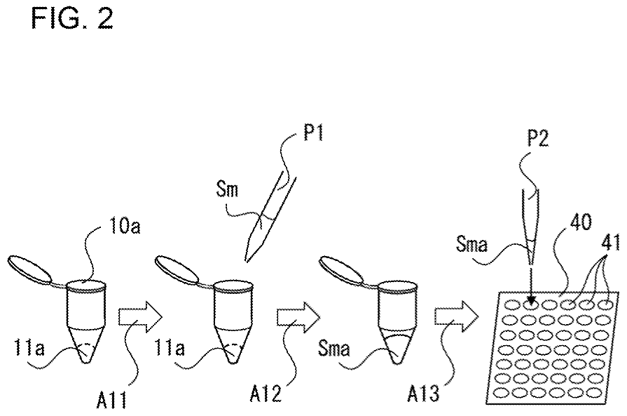

FIG. 2 is a conceptual diagram illustrating a sample preparation method using the mass spectrometry kit 1a. The case in which the additive containers 10a each contains a matrix reagent will be described later as Variations. Thus, it is assumed here that the additive containers 10a each do not contain a matrix reagent.

In the method of FIG. 2, a solution containing a sample and a matrix reagent (hereinafter referred to as “matrix-containing sample solution”) Sm is prepared in a container (not shown) other than the additive container 10a, and is then added to the additive container 10a (indicated by arrow A11). The kind of the matrix reagent is not particularly limited as long as ionization is performed appropriately, and those shown in Variations below can be used appropriately. The user of the mass spectrometry kit 1a for analysis (hereinafter merely referred to as “user”) adds the matrix-containing sample solution Sm to the additive 11a contained in the additive container 10a with, for example, a pipette P1 or a dispenser (not shown).

Then, the additive 11a and the matrix-containing sample solution are well mixed with, for example, a mixer as appropriate, thereby preparing a mass spectrometry sample Sma (indicated by arrow A12). The mass spectrometry samples Sma thus prepared are then placed at sample placement sites 41 of the sample plate 40 with, for example, a pipette P2 or a dispenser (not shown) (indicated by arrow A13). The mass spectrometry kit 1a allows the mass spectrometry sample Sma to be prepared directly by adding the solution to the additive 11a which has been dispensed. This allows a complicated operation in this preparation to be reduced.

A solvent such as a solvent 21 may be added to a matrix reagent to prepare a matrix solution, the matrix solution may then be added to an additive container 10a to prepare an additive-containing matrix solution, and a sample may be added to the prepared additive-containing matrix solution to prepare a mass spectrometry sample Sma.

A microorganism identification method by mass spectrometry will be described below as example analysis suitably using the mass spectrometry kit of this Embodiment. The mass spectrometry kit 1a is used as a microorganism identification kit.

It should be noted that the mass spectrometry kit of this Embodiment is only required to be used in the case of preparing a mass spectrometry sample using an additive, and may be used for purposes other than identification of microorganisms.

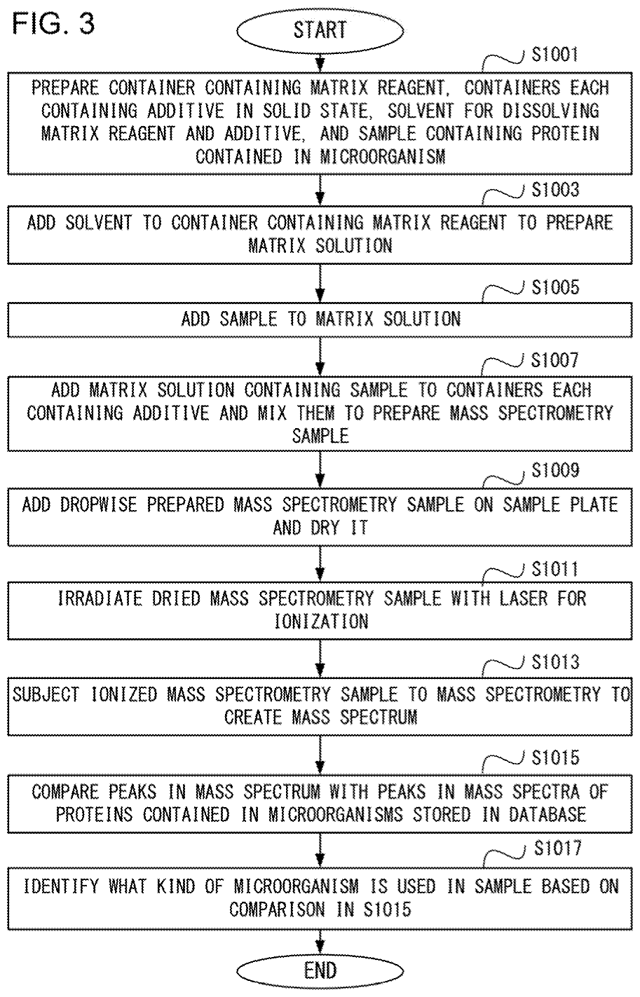

FIG. 3 is a flowchart illustrating flow of a sample preparation method, an analysis method, and a microorganism identification method, with the mass spectrometry kit of this Embodiment.

In step S1001, a container containing a matrix reagent, a plurality of containers (additive containers 10a) each containing an additive 11a in the solid state therein, a solvent for dissolving the matrix reagent and the additive 11a, and a sample containing proteins contained in a microorganism are prepared. The sample is preferably a microorganism collected from a single colony through culturing the microorganism, or an extract obtained by extraction from a microorganism with, for example, formic acid. For example, for identification of a microorganism contained in, for example, food, the sample is prepared by applying a liquid containing the microorganism extracted from food on a medium or the like, then culturing the medium, and thereafter, from the colony obtained, extracting a bacterial cell extract containing the microorganism. After the step S1001, the step S1003 is started.

In step S1003, the solvent is added to the container containing the matrix reagent, thereby preparing a matrix solution. For the effective use of the mass spectrometry kit 1a, the matrix solution is prepared preferably using the solvent 21. However, the solvent for dissolving the matrix reagent is not particularly limited as long as ionization is performed appropriately. After the step S1003, the step S1005 is started. In step S1005, the sample is added to the matrix solution. After the step S1005, the step S1007 is started.

In step S1007, the matrix solution containing the sample is added to the plurality of containers (additive containers 10a) each containing the additive 11a therein, and the sample, the matrix reagent, and the additive 11a are mixed, thereby preparing mass spectrometry samples Sma. After the step S1007, the step S1009 is started. In step S1009, the prepared mass spectrometry samples Sma are added dropwise on the sample plate 40 and then dried. After the step S1009, the step S1011 is started.

In the step S1011, the dried mass spectrometry samples Sma are irradiated with a laser, thereby ionized. The sample plate 40 is placed in a mass spectrometer, and the mass spectrometry samples Sma are irradiated with a laser from laser equipment of the mass spectrometer, thereby ionized. The wavelength and other properties of the laser are not particularly limited as long as ionization is performed appropriately, and for example, an N₂ laser (wavelength: 337 nm) can be used appropriately. After the step S1011, the step S1013 is started.

In the step S1013, the ionized mass spectrometry samples Sma are subjected to mass spectrometry to create mass spectra. How to perform mass spectrometry is not particularly limited as long as mass spectra from which microorganisms can be identified with desired accuracy can be obtained, but the mass spectrometry is preferably time-of-flight mass spectrometry for accurately detecting a protein having a molecular weight of, for example, thousands to 20000. Accordingly, the mass spectrometer which performs this mass spectrometry preferably includes a time-of-flight mass separation unit such as a flight tube.

Detection signals obtained by detecting ions in the mass spectrometry are A/D-converted with an A/D converter, and are inputted to a processor including, for example, CPU. The processor creates a mass spectrum from the A/D-converted detection signals. In the time-of-flight mass spectrometry, the processor calculates each m/z value from a time of flight using calibration data that has been obtained in advance, and the intensity detected at each m/z value is calculated to create a mass spectrum. After the step S1013, the step S1015 is started.

In step S1015, peaks in the mass spectrum are compared with peaks in mass spectra of proteins contained in a plurality of microorganisms stored in the database. The database stores data in which each kind of bacterum (e.g., the generic name and the specific epithet) is linked to the m/z of each peak observed in the mass spectum thereof. The processor determines whether each peak corresponding to the m/z indicated in the database is present in the mass spectrum obtained by the mass spectrometry of the mass spectrometry samples Sma based on an error range determined based on the accuracy of the mass spectrometry, and calculates similarity based on the determination. The similarity is a parameter indicating the degree of similarity between the mass spectrum of each kind of microorganism on the database and the mass spectrum of a microorganism corresponding to each of the mass spectrometry samples Sma. A higher degree of similarity is defined as being more similar between the mass spectra. After the step S1015, the step S1007 is started.

It should be noted that the database may contain weighting information based on the proportion and probability of observing the peak at each m/z in the mass spectrum of each kind of microorganism, and the similarity may be calculated based on the weighting information.

In step S1017, based on the comparison in step S1015, what kind of microorganism is used in the sample is identified. The processor identifies the kind of the microorganism on the database that has a calculated similarity being a certain degree or more and the highest as the microorganism contained in the sample. The identified kind of bacterium is displayed appropriately on a display such as a liquid crystal monitor. After the step S1017, the process is completed.

Classification of the microorganism to be identified is preferably made by the genera and the species, but is not particularly limited as long as the microorganism can be identified based on the difference between mass spectra. The microorganism identification method is not particularly limited as long as it is performed based on the result of the mass spectrometry of the mass spectrometry kit 1a.

By the Embodiment, the following actions and effects can be obtained.

(1) The mass spectrometry kit according to this Embodiment is a mass spectrometry kit 1a for use in mass spectrometry with ionizing a sample by MALDI, and includes an additive 11a of matrix in the solid state and a plurality of additive containers 10a each containing the additive 11a therein. This can reduce the complicated operation at the time when the mass spectrometry sample Sma is prepared using the additive 11a, thereby allowing the mass spectrometry to be performed efficiently.

(2) The mass spectrometry kit of this Embodiment further includes a solvent 21 placed in a solvent container 20 different from the additive containers 10a each containing the additive 11a therein. In this configuration, the solvent 21 which can be used for preparation of an additive solution, a matrix solution, and other solutions can be provided together. This allows the time and effort for refilling by purchasing or the like to be reduced.

(3) The sample preparation method of this Embodiment is a method for preparing a plurality of samples for use in mass spectrometry with MALDI and includes providing a plurality of additive containers 10a each containing an additive 11a in the solid state therein, and preparing mass spectrometry samples containing respective samples in the plurality of additive containers 10a using the additive 11a and the matrix reagent. This can reduce the complicated operation, thereby allowing mass spectrometry to be performed efficiently.

(4) In the sample preparation method of this Embodiment, the mass spectrometry samples Sma are prepared by adding the respective samples, a matrix reagent, and the solvent to the plurality of additive containers 10a each containing the additive 11a therein.

This allows mass spectrometry samples Sma to be prepared without dispensing the additive solution containing the additive 11a.

(5) The analysis method of this Embodiment includes preparing the mass spectrometry sample Sma by the sample preparation method described above, irradiating the mass spectrometry sample Sma with a laser to ionize, and subjecting the ionized mass spectrometry sample Sma to mass spectrometry. This can reduce the complicated operation at the time when the mass spectrometry sample Sma is prepared using the additive 11a, thereby allowing the mass spectrometry to be performed efficiently.

(6) The microorganism identification method of this Embodiment includes preparing a mass spectrometry sample Sma containing a plurality of proteins contained in a microorganism by the sample preparation method described above, irradiating the mass spectrometry sample Sma with a laser to ionize, subjecting the ionized mass spectrometry sample Sma to mass spectrometry to create a mass spectrum, comparing peaks in the mass spectrum with peaks in mass spectra of proteins contained in a plurality of microorganisms stored in a database, and identifying what kind of microorganism the above microorganism is based on the comparison. This can reduce the complicated operation at the time when the mass spectrometry sample Sma is prepared using the additive 11a for reducing noises or enhancing detection accuracy in mass spectrometry, thereby allowing the mass spectrometry to be performed efficiently.

The following Variations are also within the scope of the present invention and can be combined with the Embodiment described above. In the following Variations, parts having the same structures or functions as those of the above-mentioned Embodiment are denoted by the same reference numerals, and the descriptions of the parts are omitted as appropriate.

Variation 1

As a suitable Variation of the Embodiment, the additive containers may each contain a matrix reagent in addition to the additive 11a.

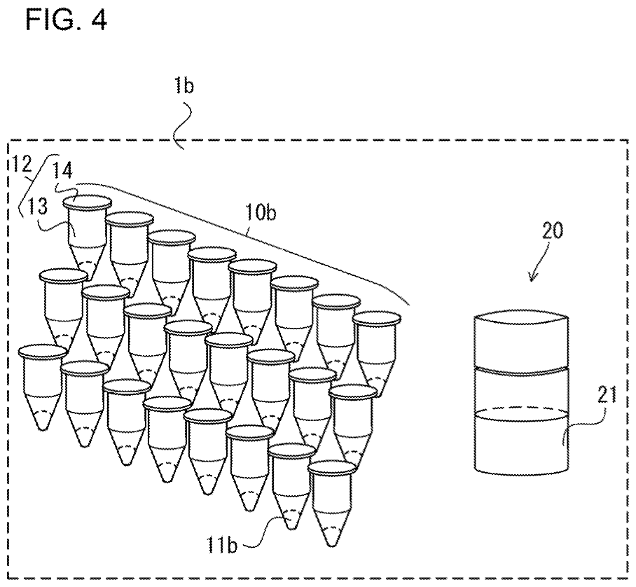

FIG. 4 is a conceptual diagram illustrating a mass spectrometry kit of this Variation. A mass spectrometry kit 1b is different from the mass spectrometry kit 1a of the Embodiment in that it includes additive containers 10b each containing a mixture 11b of a matrix reagent in the solid state and an additive 11a in the solid state.

The kind of the matrix reagent in the mixture 11b is not particularly limited as long as ionization is performed appropriately. The matrix reagent is a compound constituting a solid matrix or a liquid matrix in the mass spectrometry sample Sma. The liquid matrix is liquid at room temperature, and is, in fact, a salt. When the matrix reagent constitutes a solid matrix, the matrix reagent is preferably, 9-aminoacridine, 4-aminoquinaldine, 9-anthracenecarboxylic acid, anthranilic acid amide, caffeic acid, curcumin, 4-chloro-α-cyanocinnamic acid, α-cyano-4-hydroxycinnamic acid, sinapic acid, 1,5-diaminonaphthalene, 2,5-dihydroxybenzoic acid, 3-hydroxypicolinic acid, or trans-2-[3-(4-tert-butylphenyl)-2-methyl-polyvinylidene]malononitrile.

When the matrix reagent constitutes a liquid matrix, the matrix reagent preferably contains an amine and an organic substance with the amine serving as a proton receptor and the organic substance serving as a proton donor. In this case, it is preferred that, as the matrix reagent, a mixture obtained by mixing an amine and an organic substance that will constitute a liquid matrix in a dried and hardened state is placed in each of the additive containers 10a.

It should be noted that either the amine or the organic substance may have been placed in each of the additive containers 10a. In this case, in the preparation of the mass spectrometry sample Sma, if the amine has been placed in each of the additive container 10a, a solution containing an organic substance is prepared separately and is then added to each of the additive containers 10a, and if the organic substance has been placed in each of the additive containers 10a, a solution containing amine is prepared separately and is then added to each of the additive containers 10a.

The amine constituting the liquid matrix preferably includes a primary amine bound to a moiety such as methyl, ethyl, n-propyl, isopropyl, n-butyl, isobutyl, n-pentyl, isopentyl, n-hexyl, isohexyl, n-heptyl, isoheptyl, n-octyl, isooctyl, n-nonyl, isononyl, n-decyl, isodecyl, n-undecyl or isoundecyl, or a phenyl moiety, where nitrogen atom(s) may be substituted with an OH group.

The amine constituting the liquid matrix preferably includes a secondary or tertiary amine bound to two or three moieties which may be the same as or different from each other, selected from the group consisting of methyl, ethyl, n-propyl, isopropyl, n-butyl, isobutyl, n-pentyl, isopentyl, n-hexyl, isohexyl, n-heptyl, isoheptyl, n-octyl and isooctyl, and a phenyl moiety, where nitrogen atom(s) may be substituted with an OH group.

The amine constituting the liquid matrix preferably includes 3-aminoquinoline, pyridine, imidazole, or C- or N-alkylated imidazole derivatives.

The organic substance constituting the liquid matrix preferably includes a p-coumarate ion, 2,5-dihydroxybenzoic acid or isomers thereof (specifically 2,6-dihydroxybenzoic acid), 2-hydroxy-5-methoxybenzoic acid or isomers thereof, nicotinic acid, 3-hydroxypicolic acid, nicotinic acid, 5-chloro-2-mercaptobenzothiazole, 6-aza-2-thiothymine, trifluoromethanesulfonate, 2′,4′,6′-trihydroxyacetophenone monohydrate, 2′,6′-dihydroxyacetophenone, 9H-pyrido[3,4-b]indole, dithranol, trans-3-indoleacrylic acid, osazones, ferulic acid, 2,5-dihydroxyacetophenone, 1-nitrocarbazole, 7-amino-4-methylcoumarin, 2-(p-hydroxyphenylazo)-benzoic acid, 8-aminopyrene-2,3,4-trisulfonic acid, 2[2E-3-(4-tert-butyl-phenyl)-2-methylprop-2-enylidene]malononitrile (DCTB), 4-methoxy-3-hydroxycinnamic acid, or 3,4-dihydroxycinnamic acid.

It should be noted that a mixture of a plurality of liquid matrixes may also be used.

As shown in the following examples, when mass spectrometry samples Sma are prepared using sinapic acid as a matrix reagent, and an additive 11a containing a phosphonic acid group-containing compound such as MDPNA as an additive for the matrix reagent, the S/N ratio in mass spectrometry can be particularly enhanced, and samples can be analyzed more precisely.

Additive containers 10b each containing a mixture 11b are produced in the following manner. An additive solution containing an additive 11a at a predetermined concentration is prepared using a solvent which may be used for preparation of a matrix solution, such as a solvent 21. Then, to the additive solution, a matrix reagent is added, thereby preparing an additive-containing matrix solution. Thereafter, a predetermined amount of the additive-containing matrix solution is dispensed into each of additive containers 10b using a pipette, a dispenser or the like. The additive containers 10b into which the additive-containing matrix solution has been dispensed are dried to obtain the additive containers 10a each containing the mixture 11b in the solid state. How to dry the additive-containing matrix solution is not particularly limited. Additive container lids 13 for the additive containers 10b may be left open, or the additive containers 10a may be depressurized by, for example, a vacuum dryer.

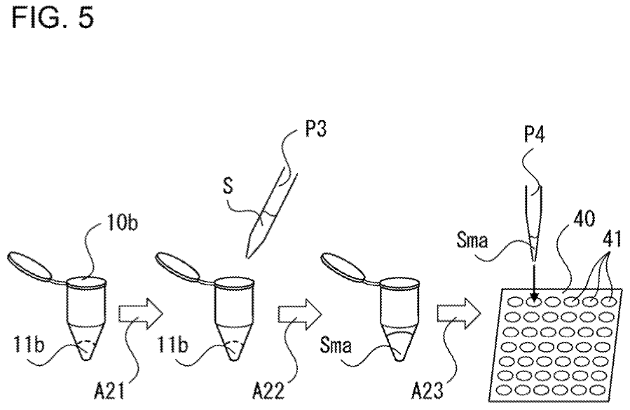

FIG. 5 is a conceptual diagram illustrating a sample preparation method using the mass spectrometry kit 1b. In the method shown in FIG. 5, a solution S containing a sample is prepared in a container (not shown) other than the additive containers 10b, is then added to the additive containers 10b (indicated by arrow A21). The user adds the solution S containing the sample to a mixture 11b contained in each of the additive containers 10b with a pipette P3 or a dispenser (not shown).

Then, the sample, the additive 11a, and the matrix reagent are well mixed with, for example, a mixer, as appropriate, thereby preparing a mass spectrometry sample Sma (indicated by arrow A22). The mass spectrometry sample Sma thus prepared is then placed at sample placement sites 41 of the sample plate 40 with, for example, a pipette P4 or a dispenser (not shown) (indicated by arrow A23). By using the mass spectrometry kit 1b in this manner, the mass spectrometry sample Sma may be prepared directly by adding the solution to the matrix reagent and the additive 11a which have been dispensed. This allows a complicated operation in this preparation to be reduced.

A microorganism identification method by mass spectrometry will be described below as example analysis suitably using the mass spectrometry kit of this Variation. The mass spectrometry kit 1b is used as a microorganism identification kit. The mass spectrometry kit of this Variation is only required to be used in the case of preparing a mass spectrometry sample using an additive, and may be used for purposes other than identification of microorganisms.

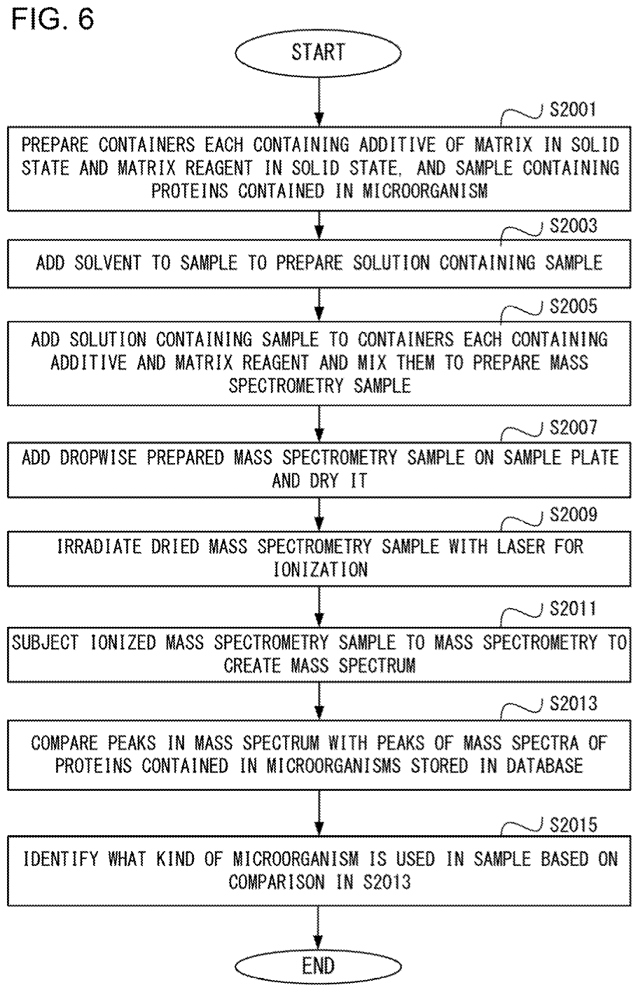

FIG. 6 is a flowchart illustrating flow of a sample preparation method, an analysis method, and a microorganism identification method, using the mass spectrometry kit of this Variation.

In step S2001, a plurality of containers (additive containers 10b) each containing an additive 11a in the solid state and a matrix reagent in the solid state therein, and a sample containing proteins contained in a microorganism are provided. After the step S2001, the step S2003 is started. In step S2003, a solvent is added to the sample to prepare a solution S containing the sample. The solution S containing the sample is prepared preferably using the solvent 21 for effective utilization of the mass spectrometry kit 1b, but the solvent to be added to the sample is not particularly limited as long as ionization is performed appropriately. After the step S2003, the step S2005 is started.

In step S2005, the solution S containing the sample is added to the plurality of containers 10b each containing the additive 11a and the matrix reagent placed therein, which is then mixed to prepare mass spectrometry samples Sma. After the step S2005, the step S2007 is started. Steps S2007 to S2015 are the same as steps S1009 to S1017 in the flowchart (FIG. 3) in the above Embodiment, and thus the description is omitted. After the step S2015, the process is completed.

In the mass spectrometry kit of this Variation, the plurality of additive containers 10b each further contain a matrix reagent that has been mixed with the additive 11a. This can further reduce the complicated operation when the mass spectrometry samples Sma are prepared using an additive 11a, thereby allowing mass spectrometry to be performed efficiently.

In the sample preparation method of this Variation, the plurality of additive containers 10b each further contain a matrix reagent in the solid state in addition to an additive 11a, and a sample and a solvent are added to the plurality of additive containers 10b each containing the matrix reagent and the additive 11a therein, to prepare mass spectrometry samples Sma. This allows mass spectrometry samples Sma to be prepared without dispensing the solution containing the matrix reagent.

Variation 2

In the above Embodiment, the mass spectrometry kit may include a matrix vial which is different from the plurality of additive containers 10a each containing an additive 11a placed therein, and a matrix reagent in the solid state placed in this matrix vial. The kind of the matrix reagent is not particularly limited and can be a substance constituting a solid matrix or a liquid matrix as shown in the above Variation.

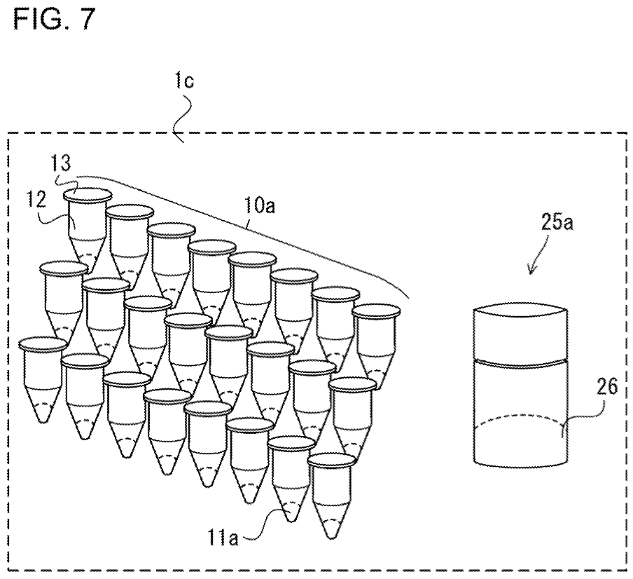

FIG. 7 is a conceptual diagram illustrating a mass spectrometry kit of this Variation. A mass spectrometry kit 1c is different from the mass spectrometry kit 1a of the Embodiment in that it includes no solvent 21 but includes a matrix vial 25a, and the matrix vial 25a contains a matrix reagent 26 in the solid state. Thus, the additive 11a and the matrix reagent 26 are collectively provided. This allows the time and effort for refilling by purchasing or the like to be reduced, and allows the amount of matrix in the mass spectrometry sample Sma to be flexibly adjusted. The shape, capacity, and the like of the matrix vial 25a is not particularly limited. The matrix reagent 26 may be divided and placed in a plurality of matrix containers as in the case of the additive 11a.

The mass spectrometry kit 1c may further include a solvent 21 and other consumables, as appropriate. The matrix vial 25a is not limited to a vial, and may be any container.

Variation 3

In the above Embodiment, the mass spectrometry kit may include no additive 11a but include a matrix reagent placed in a matrix container in the same shape as the additive containers 10a. The kind of the matrix reagent is not particularly limited and can be a substance constituting a solid matrix or a liquid matrix as shown in the above Variation.

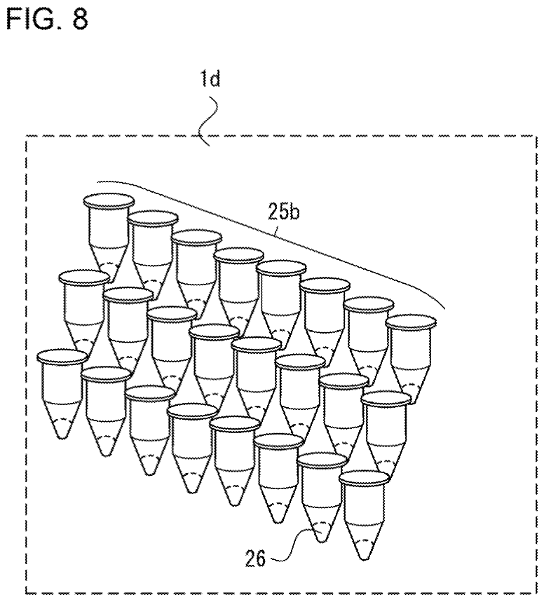

FIG. 8 is a conceptual diagram illustrating a mass spectrometry kit of this Variation. A mass spectrometry kit 1d is different from the mass spectrometry kit 1a of the Embodiment in that it includes no solvent 21 but includes a plurality of matrix containers 25b in the same shape as the additive containers 10a in the above Embodiment, and that these matrix containers 25b each contain the matrix reagent 26 in the solid state. This allows a complicated operation such as dispensing to be avoided, and a mass spectrometry sample to be prepared rapidly.

It should be noted that the mass spectrometry kit 1d may further include a container containing an additive 11a or containers each containing an additive 11a divided, a solvent 21, and other consumables, as appropriate. The matrix containers 25b may each contain either amine or an organic substance constituting a liquid matrix placed therein. In this case, a solution containing amine and a solution containing an organic substance are individually prepared and mixed together, thereby obtaining a matrix solution.

The capacity of each of the matrix containers 25b is preferably 5 mL or less, more preferably 2 mL or less, yet more preferably 1.5 mL or less, yet more preferably 1.0 mL or less, yet more preferably 0.5 mL or less, for saving space in storage.

Too small capacity of each of the matrix containers 25b causes difficulty in handling. Thus, the capacity of each of the matrix containers 25b is preferably 100 μL or more, more preferably 200 μL or more.

Although FIG. 8 shows twenty-four matrix containers 25b, the number of matrix containers 25a each containing a matrix reagent 26 therein included in the mass spectrometry kit 1d is not particularly limited.

Accordingly, the number of matrix containers 25b included in the mass spectrometry kit 1d is preferably two or more, more preferably five or more, yet more preferably 10 or more, yet more preferably 20 or more. The greater the number of matrix containers 25b included in the mass spectrometry kit 1d, the less operation is required to refill the matrix containers 25b each containing a matrix reagent 26 by purchasing or the like.

The too great number of matrix containers 25b included in the mass spectrometry kit 1d causes problems of losing activity of the matrix reagent 26 due to the long storage period, or requires large space for storage, which results in narrow space for other substances. Thus, the number of matrix containers 25b can be appropriately 1000 or less, or 500 or less.

Variation 4

In the above Embodiment and the Variations, the mass spectrometry kits 1a, 1b, 1c, and 1d are applied to a premix method in which a mass spectrometry sample containing a sample, a matrix reagent, and an additive 11a is prepared and then the mass spectrometry sample is added dropwise on a sample plate. However, the mass spectrometry kits 1a, 1b, 1c, and 1d may be applied to an on-plate method in which a sample-matrix mixture crystal is formed on a sample plate, and then an additive solution is added on the sample plate. In the mass spectrometry kits 1a, 1b, and 1c, the additive 11a or the matrix reagent 26 is dispensed. This configuration does not require freezing and thawing repeatedly, for example, in the case in which the additive 11a or the matrix reagent 26 is required to be cryopreserved, thereby allowing the additive 11a or the matrix reagent 26 to remain active for a longer period of time.

### EXAMPLES

The following shows examples, but the present invention is not limited thereby.

### Example 1: Mass Spectrometry of Protein Contained in Normal Skin Flora

<1-1. Preparation of Solution Containing Sample>

A sample containing a microorganism, obtained by wiping off the skin, was applied on a GMA medium, and cultured anaerobically at 37° C. A single colony on an agar plate was suspended in 20 μL of a 50% acetonitrile (ACN) solution containing 1% trifluoroacetic acid (TFA), thereby obtaining a bacterial cell extract.

<1-2. Production of Mass Spectrometry Kit>

A 50% aqueous ACN solution containing 1% TFA was added to sinapic acid (hereinafter referred to as “SA”), thereby preparing 20 mg/mL SA solution. A 50% aqueous ACN solution containing 1% TFA was added to MDPNA, thereby preparing 2 mg/100 mL LMDPNA solution.

(1) SA kit (Comparative Example): 5 μL of the 20 mg/mL SA solution was added to each of PCR tubes, and the solvent was then dried. That is, addition of 10 μL of a solution to each of the PCT tubes gives a 10 mg/mL SA solution.
(2) MDPNA-containing SA kit (Examples): The 20 mg/mL SA solution and 2 mg/100 mL MDPNA were mixed in equal amounts, 10 μL of the resultant mixture was added to each of PCR tubes, and the solvent was then dried. That is, addition of 10 μL of a solution gives a 10 mg/mL SA solution containing MDPNA.

<1-3. Mass Spectrometry by MALDI>

(1) 10 μL of the bacterial cell extract was added to the SA kit and mixed well, 1 μL each of the resultant mixture was added dropwise on a MALDI sample plate, and the MALDI sample plate was dried, thereby obtaining sample-matrix mixture crystals.
(2) 10 μL of the bacterial cell extract was added to the MDPNA-containing SA kit and mixed well, 1 μL each of the resultant mixture was added dropwise on a MALDI sample plate, and the MALDI sample plate was dried, thereby obtaining sample-matrix mixture crystals.

The sample-matrix mixture crystals obtained in (1) and (2) above were subjected to time-of-flight mass spectrometry in linear mode in the measurement m/z range of 3000 to 15000.

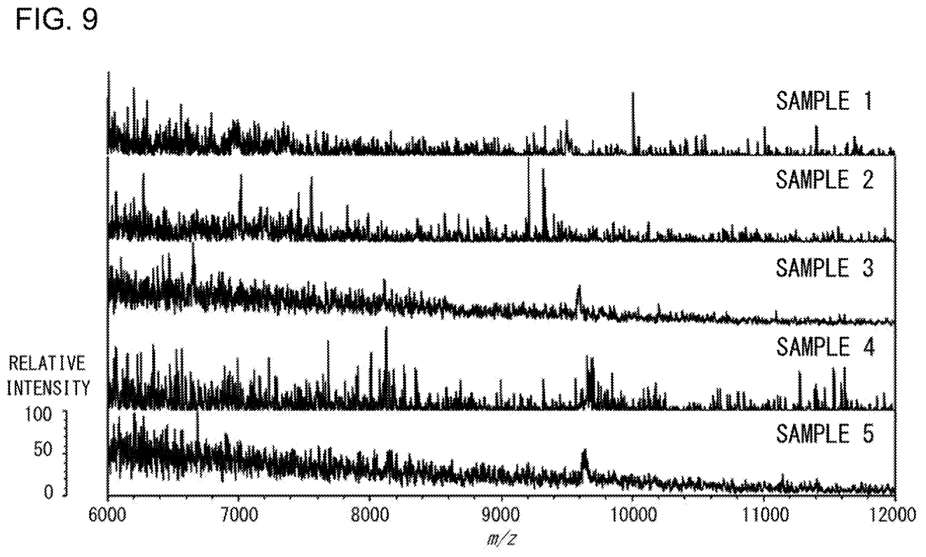

FIG. 9 shows MALDI mass spectra of samples prepared using the SA kit. In all the samples 1 to 5, noises were only detected, and no clear peak was detected.

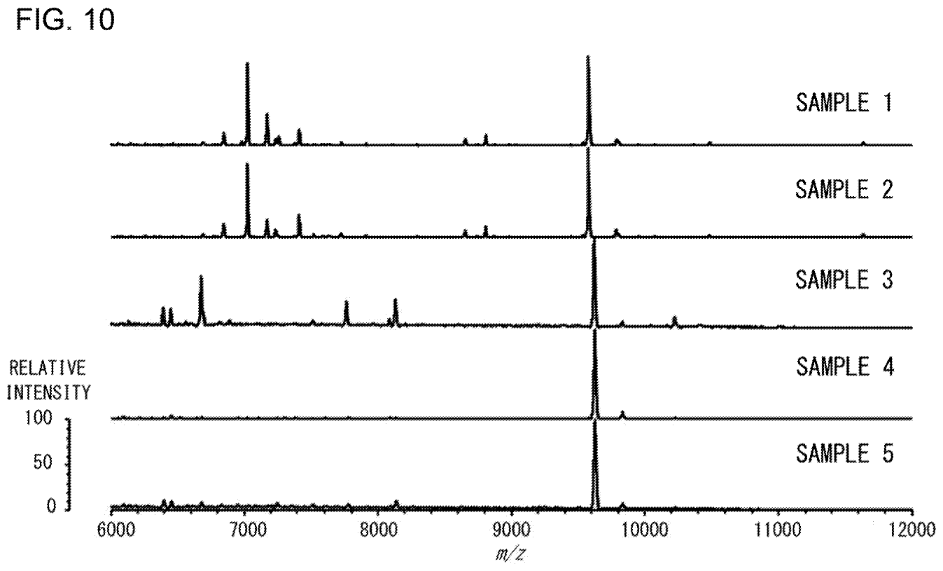

FIG. 10 shows MALDI mass spectra of the samples prepared using the MDPNA-containing SA kit. In the mass spectra of the samples prepared using the MDPNA-containing SA kit, clear peaks were detected.

### Example 2: Mass Spectrometry of Protein Contained in Lactic Acid Bacteria

<2-1. Preparation of Solution Containing Sample>

A lactic acid bacterium Lactobacillus fermentum was cultured, disrupted with zirconia beads, and centrifuged at 15000 rpm for 10 minutes to remove debris. The resultant supernatant was dissolved in a 50% aqueous ACN solution containing 1% TFA, thereby obtaining a disrupted bacterial cell solution.

<2-2. Mass Spectrometry by MALDI>

(1) The disrupted bacterial cell solution and a matrix solution (SA: 10 mg/mL) were mixed at a ratio of 1:10 to prepare a mixture, 1 μL each of the mixture was added dropwise on a MALDI sample plate, and the MALDI sample plate was dried, thereby obtaining sample-matrix mixture crystals (Comparative Example).
(2) 10 μL each of the mixture was added to a MDPNA kit prepared such that addition of 10 μL of solvent gives 1% MDPNA (1 mg/100 mL), then mixed, 1 each of the resultant mixture was added dropwise on a MALDI sample plate, and the MALDI sample plate was dried, thereby obtaining sample-matrix mixture crystals containing MDPNA (Example)

The sample-matrix mixture crystals obtained in (1) and (2) above were subjected to time-of-flight mass spectrometry in linear mode in the measurement m/z range of 4000 to 20000.

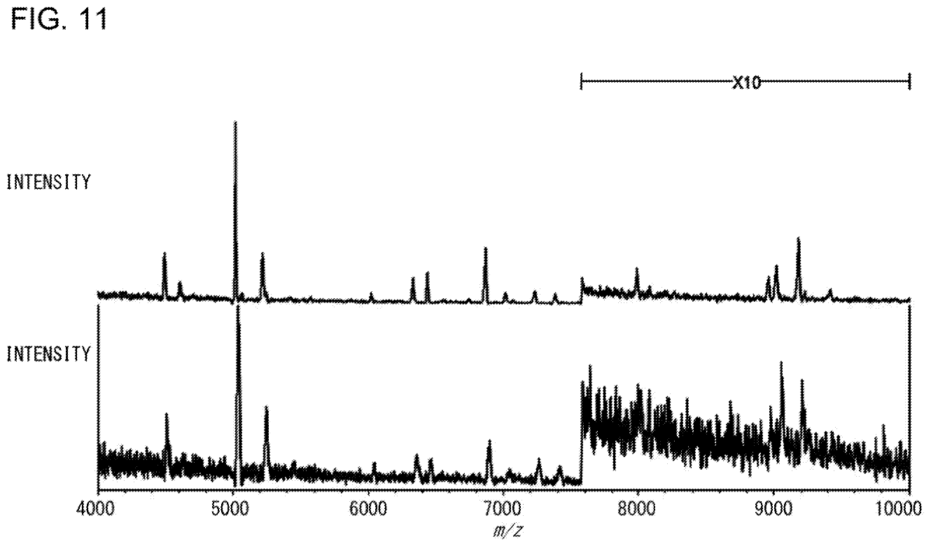

FIG. 11 shows a comparison of a MALDI mass spectrum of L. fermentum prepared using the MDPNA kit with a mass spectrum of L. fermentum prepared without using a MDPNA kit. It can be seen that the sample prepared using the MDPNA-containing kit in the upper mass spectrum shows peaks with remarkably better S/N than the control prepared using the SA kit in the lower spectrum.

The present invention is not limited by the above Embodiment. Other aspects conceivable within the scope of the technical idea of the present invention are encompassed in the scope of the present invention.

The disclosure of the following priority application is herein incorporated by reference: Japanese Patent Application No. 2018-078584 filed on Apr. 16, 2018.

### REFERENCE SIGNS LIST

1a, 1b, 1c, 1d: mass spectrometry kit; 10a, 10b: additive container; 11a: additive; 11b: mixture; 20: solvent container; 21: solvent; 25a: matrix vial; 25b: matrix container; 26: matrix reagent; 40: sample plate; 41: sample placement site; S: sample; Sm: matrix-containing sample solution; Sma: mass spectrometry sample.

1. A mass spectrometry kit for use in mass spectrometry with ionizing a sample by matrix-assisted laser desorption/ionization, the mass spectrometry kit comprising:
a matrix reagent in a solid state; and
a plurality of containers each containing the matrix reagent.

2. The mass spectrometry kit according to claim 1, wherein
a capacity of each of the plurality of containers is 100 μL or more to 5 mL or less.

3. The mass spectrometry kit according to claim 2, wherein
a capacity of each of the plurality of containers is 200 μL or more to 3 mL or less.

4. The mass spectrometry kit according to claim 1, wherein
the number of the plurality of containers is 5 or more.

5. The mass spectrometry kit according to claim 1, wherein
the plurality of containers each contain a mixture of the matrix reagent and an additive of matrix.

6. The mass spectrometry kit according to claim 1, wherein
the matrix reagent includes a substance constituting a solid matrix or a liquid matrix.

7. The mass spectrometry kit according to claim 1, further comprising:
a solvent placed in a solvent container that is different from the plurality of containers each containing the matrix reagent and/or the additive.

8. The mass spectrometry kit according to claim 1, wherein
the plurality of containers each contain a phosphonic acid group-containing compound as an additive of matrix.

9. A microorganism identification kit comprising the mass spectrometry kit according to claim 1.

10. A sample preparation method for preparing a plurality of samples for use in mass spectrometry with matrix-assisted laser desorption/ionization, the sample preparation method comprising:
providing a plurality of containers each containing a matrix reagent in a solid state therein; and
preparing mass spectrometry samples containing respective samples corresponding to the plurality of containers, using the matrix reagent.

11. The sample preparation method according to claim 10, wherein
the mass spectrometry samples are prepared by adding the respective samples and the solvent to the plurality of containers each containing the matrix reagent therein.

12. The sample preparation method according to claim 10, wherein
a mixture of the matrix reagent and an additive of matrix is placed in each of the plurality of containers, and
the mass spectrometry samples are prepared by adding the respective samples and the solvent to the plurality of containers each containing the matrix reagent and the additive therein.

13. An analysis method comprising:
preparing a mass spectrometry sample by the sample preparation method according to claim 10;
irradiating the mass spectrometry sample with a laser to ionize the mass spectrometry sample;
subjecting the ionized mass spectrometry sample to mass spectrometry.

14. A microorganism identification method comprising:
preparing a mass spectrometry sample containing a plurality of proteins contained in a microorganism by the sample preparation method according to claim 10;
irradiating the mass spectrometry sample with a laser to ionize the mass spectrometry sample;
subjecting the ionized mass spectrometry sample to mass spectrometry to create a mass spectrum;
comparing peaks in the mass spectrum with peaks in mass spectra of proteins contained in a plurality of microorganisms stored in a database; and
identify what kind of microorganism the microorganism is on the basis of the comparison.
